(19) United States
(12) Patent Application Publication
(10) Pub. No.: US 2017/0290565 A1
(43) Pub. Date: Oct. 12, 2017

(54) ULTRASOUND PROBE

(71) Applicant: Konica Minolta, Inc., Tokyo (JP)

(72) Inventors: Kiyoshi FUJII, Kanagawa (JP); Toshiharu SATO, Tokyo (JP)

(21) Appl. No.: 15/483,525

(22) Filed: Apr. 10, 2017

(30) Foreign Application Priority Data
Apr. 12, 2016 (JP) 2016079810

(51) Int. Cl. A61B 8/00; A61B 8/12; G01S 7/52

(52) U.S. Cl. CPC A61B 8/4281; G01S 7/52079; A61B 8/12; A61B 8/445

(57) ABSTRACT

An ultrasound probe of the present invention includes: a piezoelectric element configured to transmit and receive an ultrasonic wave; a casing that houses the piezoelectric element; and an acoustic medium liquid that fills a space between the piezoelectric element and the casing, and contains an aromatic compound or a substituted derivative thereof

### CROSS REFERENCE TO RELATED APPLICATIONS

This application is entitled to and claims the benefit of Japanese Patent Application No.2016-079810, filed on Apr. 12, 2016, the disclosure of which including the specification, drawings and abstract is incorporated herein by reference in its entirety.

### BACKGROUND OF THE INVENTION

### 1. Field of the Invention

The present invention relates to an ultrasound probe for use in ultrasound diagnosis.

### 2. Description of Related Art

The use of hydrocarbon oils having a kinematic viscosity of 20 m/s or lower or viscosity of 20 mPs/s or lower is a characteristic of acoustic media used for related-art mechanical scanning-type ultrasound probes (e.g., see Japanese Patent Application Laid-Open No. 2001-299748).

Ultrasound diagnostic apparatus includes an ultrasound probe configured to be connected to or to communicate with the ultrasound diagnostic apparatus. The ultrasound diagnostic apparatus can obtain ultrasound diagnostic images of tissue shapes, tissue movements, or the like by a simple operation of putting the probe on a body surface or inserting the probe into a body, and can conduct tests repeatedly due to its high safety. The ultrasound probe includes a tip housing section that encloses, for example, piezoelectric elements that transmit and receive ultrasonic waves, and a grip section for holding the whole ultrasound probe to operate it.

The piezoelectric elements, configured to be connected to or to communicate with the ultrasound diagnostic apparatus, convert electrical signals (transmission signals) from the ultrasound diagnostic apparatus into ultrasonic signals, transmit the ultrasonic signals, receive ultrasonic waves reflected inside a living body, convert the ultrasonic waves into electrical signals (reception signals), and transmit the reception signals converted as the electrical signals to the ultrasound diagnostic apparatus.

There is known an ultrasound probe that scans a subject by mechanically rotating or swinging piezoelectric elements. In the ultrasound probe, piezoelectric elements and a mechanism section for rotating or swinging the piezoelectric elements are disposed inside the tip housing section.

A surface of the tip housing section, which faces wave-transmitting/receiving surfaces of the piezoelectric elements, is provided with a window made of a material that readily transmits ultrasonic waves. A gap between the wave-transmitting/receiving surfaces of the piezoelectric elements and the window is filled with an acoustic medium liquid having an acoustic impedance close to that of a living body.

The acoustic medium liquid is used for effectively transmitting and receiving ultrasonic waves by acoustically matching the wave-transmitting/receiving surfaces of the piezoelectric elements and the window. Thus, only a gap between the wave-transmitting/receiving surfaces of the piezoelectric elements and the window may theoretically be filled with the acoustic medium liquid. However, practically, filling only the gap with the acoustic medium liquid is difficult. Thus, generally, such filling is implemented by a method of closing a space where the piezoelectric elements are enclosed in a liquid-tight manner, and filling the sealed space with the acoustic medium liquid.

As acoustic medium liquids for use in mechanical scanning-type ultrasound probes, hydrocarbon oils are widely used in the related art. For example, a hydrocarbon oil having a kinematic viscosity of 20 mm²/s or lower is used (Japanese Patent Application Laid-Open No. 2001-299748). Also, a hydrocarbon oil having a viscosity of 10 to 20 mPa-s is used in an attempt to improve attenuation of ultrasonic signals in a high-viscosity acoustic medium liquid (Japanese Patent Application Laid-Open No. 2013-198645).

However, such hydrocarbon oils tend to have lower densities as the viscosities become lower. Accordingly, it is preferable to use hydrocarbon oils having low viscosities as acoustic medium liquids from the viewpoint of suppressing attenuation of ultrasonic signals or occurrence of image noise. However, in this case, the densities of the acoustic medium liquids also become low. Hydrocarbon oils generally have densities of lower than 0.9, and low-molecular-weight hydrocarbon oils with low viscosity have further lower densities.

When propagating in different media, ultrasonic waves are reflected in proportion to differences in acoustic impedances between the media. Acoustic impedance is a product of density and acoustic velocity of a medium. Thus, when hydrocarbon oils having low viscosities are used as acoustic medium liquids from the above-mentioned viewpoint, the acoustic impedances also become low. Hydrocarbon oils generally have acoustic velocities of 1400 to 1450 m/s. Accordingly, acoustic impedances of the hydrocarbon oils are generally 1.2 MRayls, which is a significantly different value from the acoustic impedance (about 1.53 MRayls) of a living body.

Ultrasonic waves transmitted from piezoelectric elements (first transmission) propagate inside a living body in contact with a window via an acoustic medium liquid and the window. When there is a mismatch of acoustic impedances between the acoustic medium liquid and a living body as described above, the ultrasonic waves transmitted from the piezoelectric elements are reflected on a surface of the living body in proportion to differences occurred in acoustic impedances between the acoustic medium liquid and the living body. The reflected signals are propagated in the opposite direction to the original transmission direction, reflected again on the surfaces of the piezoelectric elements, and transmitted again to the living body through the acoustic medium liquid (second transmission). The above phenomenon in which the reflected signals of the first transmission generate second or later transmission of the ultrasonic waves is called multiple reflections.

Ultrasonic waves transmitted to a living body are reflected at boundaries between different acoustic impedances, such as tissue boundaries inside the living body, and received as echoes by piezoelectric elements via a window and an acoustic medium liquid. When the second transmission, delayed from the first transmission, occurs due to a mismatch of acoustic impedances between the acoustic medium liquid and the living body, received echoes of the second transmission become multiple reflection noise (artifacts) by being superimposed on an ultrasound diagnostic image of the living body which has been generated with received echoes of the original first transmission.

Thus, acoustic medium liquids known in the art have a problem in which noise (artifacts) due to multiple reflections tends to occur, thereby lowering accuracy of ultrasound diagnostic images.

Further, an acoustic medium liquid is covered with a window, and the above-mentioned multiple reflections actually occur between the acoustic medium liquid and an inner surface of the window. Meanwhile, windows of mechanical scanning-type ultrasound probes are generally composed of a material, such as polymethylpentene, having an acoustic impedance close to that of a living body. Thus, in the above description, for the purpose of simplification, the inner surface of the window is explained as the surface of the living body, assuming that the window and the living body have the same acoustic impedance.

### SUMMARY OF THE INVENTION

To solve at least one of the above-mentioned problems, an ultrasound probe reflecting one aspect of the present invention includes: a piezoelectric element configured to transmit and receive an ultrasonic wave; a casing that houses the piezoelectric element; and an acoustic medium liquid that fills a space between the piezoelectric element and the casing, in which the acoustic medium liquid contains an aromatic compound or a substituted derivative thereof.

### BRIEF DESCRIPTION OF DRAWINGS

The present invention will become more fully understood from the detailed description given hereinbelow and the appended drawings which are given by way of illustration only, and thus are not intended as a definition of the limits of the present invention, and wherein:

FIG. 1 is an external perspective view of ultrasound diagnostic apparatus using an ultrasound probe;

FIG. 2 is a sectional view illustrating the whole structure of an ultrasound probe;

FIG. 3 is an enlarged sectional view illustrating a tip housing section; and

FIGS. 4A, 4B, and 4C are graphs showing relationships between driving voltage and the number of rotations of a motor.

### DESCRIPTION OF THE PREFERRED EMBODIMENTS

In the following, an embodiment of the present invention will be described in reference to the drawings.

[Ultrasound Diagnostic Apparatus]

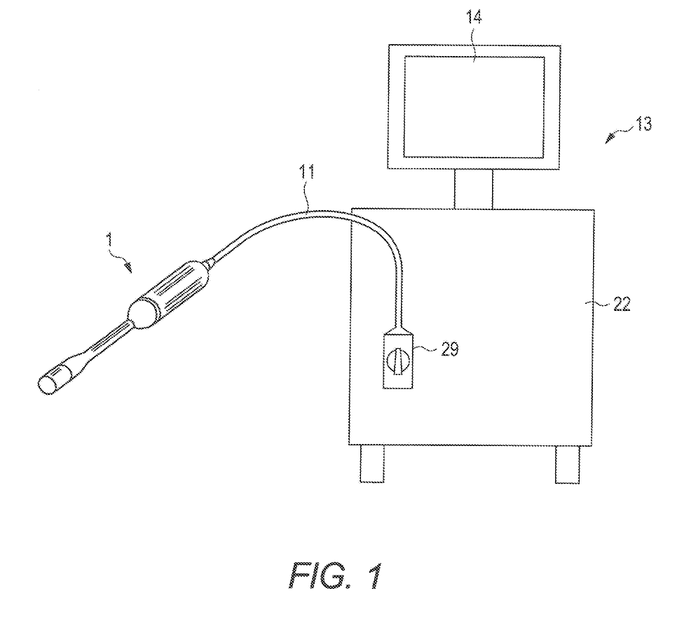

FIG. 1 is an external perspective view of ultrasound diagnostic apparatus 13 using ultrasound probe 1 according to an embodiment of the present invention.

Ultrasound diagnostic apparatus 13 includes ultrasound diagnostic apparatus body 22, connector section 29, and display 14.

Ultrasound probe 1 is connected to ultrasound diagnostic apparatus 13 through cable 11 connected to connector section 29.

Electrical signals (transmission signals) from ultrasound diagnostic apparatus 13 are transmitted to piezoelectric elements of ultrasound probe 1 through cable 11. The piezoelectric elements will be described hereinafter. The transmission signals are converted into ultrasonic waves at the piezoelectric elements, and transmitted to inside a living body The transmitted ultrasonic waves are reflected by tissues and the like inside the living body, and part of the reflected waves are received again by the piezoelectric elements, converted into electrical signals (reception signals), and transmitted to ultrasound diagnostic apparatus 13. The reception signals are converted into image data at ultrasound diagnostic apparatus 13, and shown on display 14.

In the following, ultrasound probes will be described in detail.

[Ultrasound Probe]

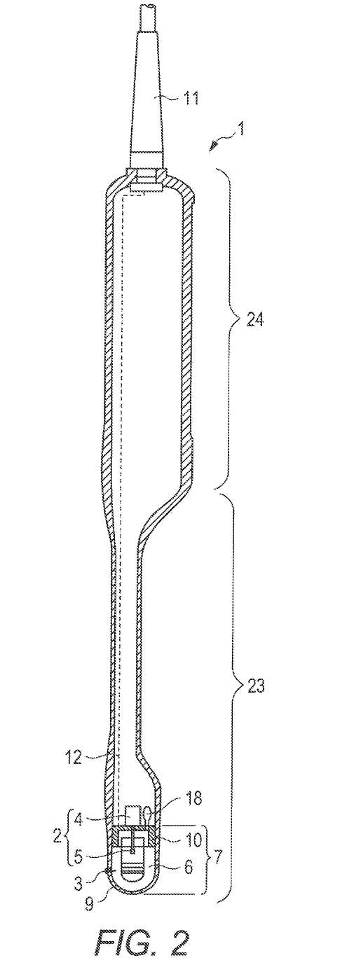

FIG. 2 is a sectional view illustrating one example of the whole structure of ultrasound probe 1. Ultrasound probe 1, used for ultrasound diagnosis, is a body cavity insertion-type probe that can be partially inserted into a body cavity of a subject and scan inside the body cavity with ultrasonic waves.

As illustrated in FIG. 2, ultrasound probe 1 includes insertion section 23 having tip housing section 7 to be inserted into a body cavity, and grip section 24 to be held by an operator outside the body cavity. Cable 11 to be connected to ultrasound diagnostic apparatus body 22 is also provided. More than one signal line 12 is pulled out of housing section 7 through the inside of insertion section 23 and grip section 24, and connected to cable 11.

Such body cavity insertion-type probes are often used by inserting into a body cavity of a subject. However, some ultrasound probes are generally also used by putting on a body surface without inserting into a body cavity of a subject. Ultrasound probes according to the present invention are not limited to a body cavity insertion type.

Although ultrasound probe 1 is configured to be connected to ultrasound diagnostic apparatus 13 through cable 11, it may also be configured to be connected with ultrasound diagnostic apparatus 13 through wireless communications without providing a cable.

In the following, tip housing section 7 will be described in detail.

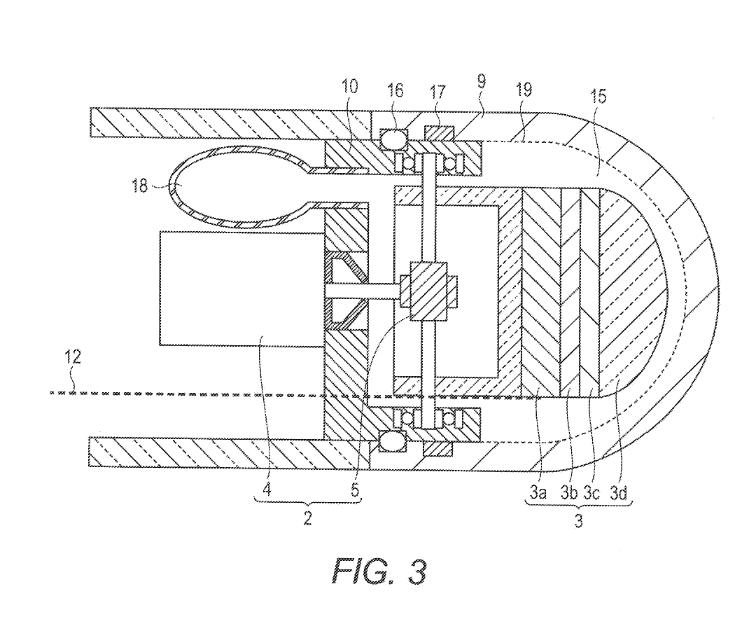

FIG. 3 is an enlarged sectional view of tip housing section 7 illustrated in FIG. 2. Tip housing section 7 is configured by bonding window 9, which constitutes part of a casing for ultrasound probe 1, and frame 10 as a holding member. Tip housing section 7 includes piezoelectric element unit 3, swinging mechanism section 2 for holding and swinging the piezoelectric element unit 3, and acoustic medium liquid storage space portion 15 filled with acoustic medium liquid 6 for propagation of ultrasonic signals.

Window 9 is composed of a material, such as polymethylpentene, having an acoustic impedance close to that of a living body.

Frame 10 is sealed with sealing member 16, such as an O-ring or a gasket, and adhesive 17 or the like, so as to come into tight contact with an inner wall of window 9, and seals tip housing section 7 in a liquid-tight manner. Frame 10 can be made of a metal or a resin, for example. As a metal frame, a frame made of aluminum, for example, can be used. As a resin frame, it is desirable to use a frame made of a resin that does not swell under the presence of acoustic medium liquid 6 as described hereinafter. In addition, frame 10 is provided with wiring holes (not shown) for passing more than one signal line 12 described above through. In order to maintain a tightly closed state of tip housing section 7, signal lines 12 and frame 10 in the wiring holes are sealed in a liquid-tight manner with an adhesive or the like.

As illustrated in FIG. 3, piezoelectric element unit 3 is configured by stacking backing layer 3a , piezoelectric element 3b , acoustic matching layer 3c , and acoustic lens 3d.

Backing layer 3a , provided on surfaces of piezoelectric element 3b on the side opposite to a living body, supports piezoelectric element 3b and absorbs ultrasonic waves transmitted to an opposite side of piezoelectric element 3b to a living body side. As a material of backing layer 3a , natural rubber, an epoxy resin, a thermoplastic resin, or the like can be used.

Piezoelectric element 3b is a layer composed of piezoelectric materials. Examples of the piezoelectric materials include lead zirconate titanate (PZT), piezoelectric ceramics, lead zincate niobate titanate (PZNT), and magnesate niobate titanate (PMNT). Piezoelectric element 3b has a thickness of 0.05 to 0.4 mm, for example. Electrodes (not shown) for applying voltage to piezoelectric element 3b are provided on surfaces of piezoelectric element 3b on both a living body side and the opposite side. The electrodes are connected to signal lines 12, and transmit electrical signals to and receive electrical signals from piezoelectric element 3b.

Acoustic matching layer 3c is a layer for matching acoustic characteristics between piezoelectric element 3b and acoustic lens 3d , and has an acoustic impedance approximately intermediate between those of piezoelectric element 3b and acoustic lens 3d . Acoustic matching layer 3c may be a single layer or laminated layers. However, from the viewpoint of adjusting acoustic characteristics, a laminate of more than one layer with different acoustic impedances is preferable (e.g., two or more layers, more preferably four or more layers), and it is more preferable to set an acoustic impedance of each layer so as to become closer to the acoustic impedance of acoustic lens 3d stepwise or continuously towards acoustic lens 3d . Each layer of acoustic matching layer 3c can be bonded with adhesives (e.g., epoxy adhesives) typically used in the art.

Acoustic matching layer 3c can be composed of various materials. For example, aluminum, aluminum alloys, magnesium alloys, Macor glass, glass, fused quartz, copper-graphite, and resins can be used. Examples of the resins include polyethylene, polypropylene, polycarbonates, ABS resin, AAS resins, AES resin, nylons, polyphenylene oxide, polyphenylene sulfide, polyphenylene ethers, polyether ether ketones, polyamide-imides, polyethylene terephthalate, epoxy resins, and urethane resins.

Acosutic lens 3d is composed of, for example, a flexible polymeric material having an acoustic impedance approximately intermediate between those of acoustic matching layer 3c and a living body, and used for focusing ultrasound beams by refraction, thereby enhancing resolution. Examples of the flexible polymeric materials include a silicone-based rubber, a butadiene-based rubber, a polyurethane rubber, an epichlorohydrin rubber, and ethylene-propylene copolymer rubber formed by copolymerizing ethylene and propylene. Among them, a silicone-based rubber and a butadiene-based rubber are preferable, and a silicone rubber of a silicone-based rubber and butadiene rubber of a butadiene-based rubber are particularly preferable.

Swinging mechanism section 2 includes transmission mechanism section 5 that holds and swings piezoelectric element unit 3, and motor 4 that drives rotation of a gear (transmission mechanism) inside transmission mechanism section 5. Accordingly, scanning with ultrasonic signals can be performed by swinging piezoelectric element unit 3 in tandem with the rotation of the gear (transmission mechanism) inside the transmission mechanism section 5. Together with or instead of swinging mechanism section 2 that holds and swings piezoelectric element unit 3, a rotating mechanism section (not shown) that holds and rotates piezoelectric element unit 3 may be provided. Further, although the gear is used as a transmission mechanism in transmission mechanism section 5 for swinging piezoelectric element unit 3, a timing belt, a wire, or the like can be used as the transmission mechanism other than the gear.

Acoustic medium liquid storage space portion 15 is a space closed with window 9 and frame 10 in a liquid-tight manner, and retains acoustic medium liquid 6.

Ultrasonic waves transmitted from piezoelectric element 3b propagate through each medium of acoustic matching layer 3c , acoustic lens 3d , acoustic medium liquid 6, and window 9 in this order, and reach a living body. The ultrasonic waves reflected by tissues inside the living body propagate through each medium in the reverse order, and are received by piezoelectric element 3b.

In the following, acoustic medium liquid 6 will be described in detail.

As mentioned above, since acoustic medium liquid 6 is positioned on transmitting/receiving paths of ultrasonic waves, the acoustic characteristics are important.

An acoustic impedance is one aspect of the acoustic characteristics of liquids. As already mentioned, ultrasonic signals are reflected in proportion to differences in acoustic impedances. Accordingly, materials for acoustic medium liquid 6 and window 9, both of which are present on propagation paths of ultrasonic signals transmitted from piezoelectric element 3b towards a living body, desirably have closer acoustic impedances to that of a living body as possible.

Attenuation characteristics of ultrasonic signals are also important as one aspect of the acoustic characteristics of acoustic medium liquid 6. High attenuation of ultrasonic signals in acoustic medium liquid 6 lowers sensitivity of ultrasound probes and causes problems, such as smaller testing depth in ultrasound diagnosis and lower brightness of images, and consequently lowers accuracy in ultrasound diagnostic images. Thus, low attenuation of ultrasonic signals is required for acoustic medium liquid 6.

From the viewpoint of the above-mentioned two acoustic characteristics, an aromatic compound is used as acoustic medium liquid 6 in the embodiment. The aromatic compound used in the embodiment is an oily substance having at least one aromatic ring without any other particular restrictions. The number of aromatic rings is preferably 1 to 4, and more preferably 1 or 2, since the viscosity becomes higher when the number is 5 or greater. The aromatic rings may be fused rings or heterocyclic rings, as well as monocyclic rings.

As the aromatic compound used in the embodiment, for example, an aromatic compound having an alkyl group bonded on the aromatic ring can be used. Examples of the aromatic compounds having an alkyl group bonded on the aromatic ring include an alkylbenzene, an alkylnaphthalene, various derivatives thereof, and the like. As alkylbenzene derivatives, those having multinuclear structures in which more than one alkylbenzene is connected through a single bond, or a divalent group, such as an alkylene group, an ether group, an ester group, a carbonate group, a carbonyl group, or a sulfonyl group, may be used. Their substituted derivatives may also be used. An alkyl group or a substituent group bonded on the aromatic ring of the aromatic compound or a derivative thereof has the number of carbon atoms of 1 to 30, preferably 4 to 25.

The aromatic compound used in the embodiment may have a double bond or a cyclic structure formed by further bonding the carbon atoms that are not forming the aromatic ring. For example, an alkylated biphenyl, a polyphenyl-substituted hydrocarbon, or a styrene oligomer can be used.

Thus, examples of the aromatic compounds used in the embodiment can be represented by an aromatic compound or a substituted derivative thereof having a structure of General Formula 1.

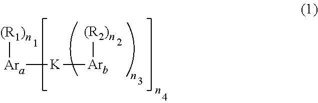

In general formula 1, Arₐ and Ar_{b} are each independently an aromatic ring; ni is an integer of 0 to 4, preferably 1 to 3; n₂ is an integer of 0 or 1 to 3, preferably 1 or 2; n₃ is an integer of 1 to 3, preferably 1 or 2, and particularly preferably 1; n₄ is an integer of 0, 1, or 2 (when n₄ is equal to 0, n₁ is not equal to 0; when n₄ is not equal to 0, (n₁+n₂) is not equal to 0).

K is a linking group selected from the following 1) to 3):

1) a single bond,

2) a divalent group selected from the group consisting of —O—, —SO₂—, —O—(C═O)—O—, —(C═O)—, —RL-O—, —O—RL-, —O—C(═O)—RL—, —C(═O)—O—RL-, —(C═S)—, —(C═O)—O—, —NRM-, —S—, —(C═O)—NRM-, and —NRM-(C═O)—, in which RL represents an alkylene group, an alkenylene group, an alkynylene group, or a cycloalkylene group, RM represents a hydrogen atom or an alkyl group, and the divalent group is preferably an oxygen atom, and

3) a di-, tri-, or tetravalent (preferably divalent) C₁₋₁₂ (preferably 1 to 4, and particularly preferably 1) saturated hydrocarbon group or a substituted group thereof R₁ and R₂ are each independently a C₁₋₃₀ (preferably 4 to 25) alkyl group or a substituted group thereof, and may contain an ether bond. R₁, R₂, K, and Ar_{b} may each have more than one structure.

In general formula 1, when more than one R₁ group is bonded to Arₐ, the R₁ groups may be the same or different. Similarly, when more than one R₂ group is bonded to Ar_{b}, the R₂ groups may be the same or different. Further, when n₄ is 2, two K bonded to Arₐ may be the same or different. Similarly, when n₃ is 2 or 3, Ar_{b} groups may be the same or different.

An aromatic compound represented by the structure of general formula 1 may contain an ether bond in a ratio of ⅓ or lower, preferably ⅕ or lower, to the total number of the carbon atoms. Within the above oxygen atom content, R₁ and R₂ may be each independently an alkyl group, an alkyl group having oxygen atom(s) at the terminal(s) or inside, or a substituted group thereof

Further, in an aromatic compound represented by general formula 1, ⅓ or lower, or preferably ⅕ or lower, of the hydrogen atoms based on the total number of the hydrogen atoms may be replaced with a polar group, such as an amino group (-NRR'), an anil group, an acyloxy group, a carboalkoxyl group, or a nitrile group.

Representative examples of the aromatic compounds having the structure of general formula 1 include benzyltoluene, 1-phenyl-1-xylylethane, 1-(2-ethylphenyl)-1-phenylethane, and 1-(4-ethylphenyl)-1-phenylethane, respectively represented by Chemical Formulas (2) to (5).

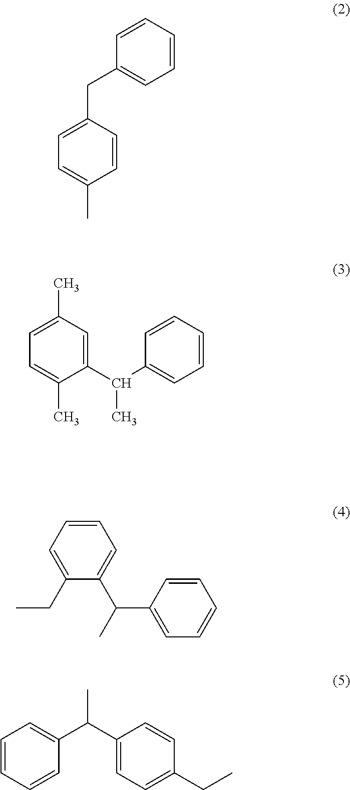

Further, acoustic medium liquid 6 used in the embodiment may be a mixture of two or more types of aromatic compounds, or a mixed oil where part of, preferably ⅔ or less or more preferably ½ or less, aromatic compounds are replaced with nonaromatic compounds (e.g., hydrocarbon oils).

Table 1 shows the acoustic characteristics of representative aromatic compounds.

TABLE 1
Acoustic characteristics and physical properties
of representative aromatic compounds and a hydrocarbon oil

| | | 1-Phenyl-1- | |
|---|---|---|---|
| | | xylylethane, | |
| | | 1-Phenyl-1- | |
| | | ethylphenylethane, | Hydrocarbon |
| Item | Benzyltoluene | or a mixture thereof | oil |
| Density (kg/m³) | 1.00 | 0.989 | 0.85 |
| Acoustic velocity | 1497 | 1540 | 1400 |
| (m/s) | | | |
| Acoustic impedance | 1.50 | 1.52 | 1.19 |
| (MRayl) | | | |
| Kinematic viscosity | 2.6 | 5.2 | 15 |
| (mm²/s) | | | |
| Ultrasonic | 0.016 | 0.067 | 1.19 |
| attenuation | | | |
| (dB/mm, at 5 MHz) | | | |
| Boiling point (° C.) | 291 | 302 | — |
| Saturated vapor | 8.3 | 8.3 | — |
| pressure (kPa) | | | |

The representative aromatic compounds shown in Table 1 have a density of 1.00 or 0.99, which is a large value compared with a density of less than 0.9 for common mineral oils or so-called liquid paraffin or linear hydrocarbon oils. Further, the aromatic compounds have an acoustic velocity of 1497 or 1540 m/s at ambient temperature, which is an extremely close value to the acoustic velocity of a living body (about 1530 m/s). Since acoustic impedance is a product of density and acoustic velocity of the medium, the acoustic impedances of the aromatic compounds are about 1.5 MRayls, which is an extremely close value to the acoustic impedance of a living body (about 1.53 MRayls). Therefore, a mismatch of acoustic impedances between acoustic medium liquid 6 and a living body actually between acoustic medium liquid 6 and window 9) is eliminated.

Moreover, attenuation characteristics of ultrasonic signals in the aromatic compounds are 0.016 and 0.067 dB/mm (ultrasonic signals at 5 MHz), which are extremely low values. Accordingly, lowering in sensitivity of ultrasound probes due to attenuated ultrasonic signals can be suppressed.

Meanwhile, as described above, linear hydrocarbon oils used as acoustic medium liquids in the related art tend to have lower densities as the viscosities become lower. Further, according to independent measurement results by the present inventors, the ultrasonic attenuation becomes lower as the viscosities become lower. Accordingly, when a hydrocarbon oil having a low viscosity is used for the purpose of low attenuation of ultrasonic signals in acoustic medium liquid 6, the acoustic impedance of acoustic medium liquid 6 becomes more different from the acoustic impedance of a living body as the density becomes lower. There is a problem of such a trade-off between attenuation characteristics of ultrasonic signals and acoustic impedance. In contrast, aromatic compounds have both low viscosities and high densities. Accordingly, by using an aromatic compound as acoustic medium liquid 6, low attenuation of ultrasonic signals in acoustic medium liquid 6 and an acoustic impedance close to that of a living body can be sought to achieve simultaneously.

From the viewpoint of the above-mentioned acoustic characteristics, an aromatic compound is suitable for acoustic medium liquid 6 of a mechanical scanning-type ultrasound probe.

Further, mechanical characteristics are also important as one aspect of the acoustic characteristics of liquids. In the following, mechanical characteristics of acoustic medium liquid 6 will be described.

Mechanical scanning-type ultrasound probes perform scanning with ultrasonic waves by mechanically rotating or swinging piezoelectric element unit 3 in acoustic medium liquid 6. Accordingly, acoustic medium liquid 6 having a high kinematic viscosity increases mechanical load, thereby making high-speed scanning difficult.

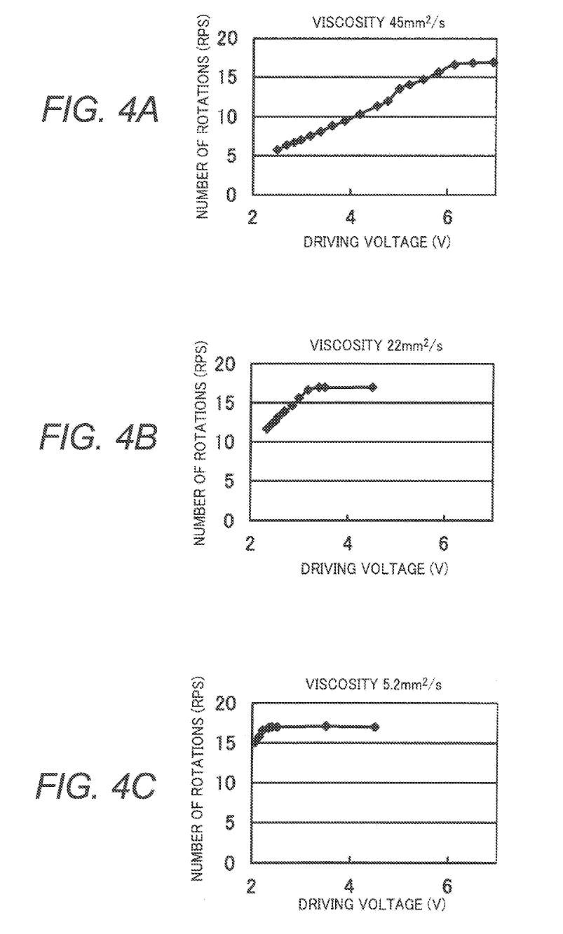

For example, FIGS. 4A, 4B, and 4C are graphs showing relationships between driving voltage and the number of rotations of motor 4. FIGS. 4A, 4B, and 4C show experimental results under environments at 40° C. and a viscosity of 45, 22, or 5.2 mm²/s. The number of rotations of a motor is preferably 17 RPS or greater, since excessively small number of rotations lowers a frame rate of ultrasound diagnostic images and damages real-time performance. As seen in FIG. 4A, a driving voltage of 6.3 V or higher is necessary for the number of rotations of a motor to reach 17 RPS under an environment at a viscosity of 45 mm²/s. Such a high voltage results in increased power consumption of a motor, and a problem in which temperature rise at an ultrasound probe due to heat generated from the motor could cause an uncomfortable feeling or a burn to a patient.

In contrast, the representative aromatic compounds have a kinematic viscosity of 2.6 or 5.2 mm²/s (at 40° C.) as shown in Table 1. As seen in FIGS. 4B and 4C, the number of rotations can reach 17 RPS at a driving voltage lower than 6.3 V (each 3.2 V or 2.3 V), since mechanical load decreases under environments at lower viscosities of 22 and 5.2 mm²/s. A driving voltage of 3.2 V or lower suppresses temperature rise at an ultrasound probe and eliminates a risk of a burn. For this reason, as acoustic medium liquid 6, a substance having low viscosity and small mechanical load on piezoelectric element unit 3 is preferably used, and a substance having a viscosity of 22 mm²/s or lower at 40 C is particularly desirable.

Also from the viewpoint of the above-mentioned mechanical characteristics, an aromatic compound is suitable for acoustic medium liquid 6 of a mechanical scanning-type ultrasound probe.

Further, stability is also important as one aspect of the acoustic characteristics of liquids. In the following, the stability of acoustic medium liquid 6 will be described.

Since acoustic medium liquid 6 is sealed in an ultrasound probe, the stability is important from the viewpoint of the maintenance of the ultrasound probe. Acoustic medium liquid 6 having a low boiling point tends to vaporize and generate air bubbles in acoustic medium liquid 6 sealed in the ultrasound probe. Trapped air bubbles and the like in acoustic medium liquid 6 result in interrupted propagation of ultrasonic waves. Therefore, acoustic medium liquid 6 is required to be less likely to undergo a liquid-to-gas phase change and to have stable properties over time.

As shown in Table 1, the representative aromatic compounds have a high boiling point of about 300° C. and a high saturated vapor pressure of 8.3 kPa (at 200° C.). Because of this, the generation of air bubbles as mentioned above is minimized, thereby eliminating causes of interrupted propagation of ultrasonic waves.

Also from the viewpoint of the above-mentioned stability, an aromatic compound is suitable for acoustic medium liquid 6 of a mechanical scanning-type ultrasound probe.

As described above, an aromatic compound is suitable for acoustic medium liquid 6 of a mechanical scanning-type ultrasound probe from the viewpoint of the acoustic characteristics, mechanical characteristics, and stability. The use of aromatic compounds as acoustic medium liquid 6 can improve a mismatch of acoustic impedances between acoustic medium liquid 6 and a living body (precisely between acoustic medium liquid 6 and window 9), and obtain high-quality ultrasound diagnostic images with suppressed artifacts caused by multiple reflections.

As already described, although acoustic medium liquid 6 is filled in acoustic medium liquid storage space portion 15 closed in a liquid-tight manner, acoustic medium liquid 6 generally expands and contracts according to the environmental temperature. Expanded acoustic medium liquid 6 may increase an internal pressure of acoustic medium liquid storage space portion 15 and cause problems, such as cracking and liquid leakage.

Further, air bubbles may also be trapped during a process for sealing acoustic medium liquid 6 in acoustic medium liquid storage space portion 15. The presence of such air bubbles between piezoelectric element unit 3 and window 9 may cause interrupted propagation of ultrasonic waves, and result in a problem in which clear ultrasonic tomographic images cannot be obtained due to attenuated or reflected ultrasonic signals by the air bubbles.

For the purpose of preventing the problem, reservoir 18 connected to acoustic medium liquid storage space portion 15 for absorbing expansion and contraction of acoustic medium liquid 6 may be installed outside acoustic medium liquid storage space portion 15, as shown in FIG. 3.

A material for reservoir 18 is preferably a fluororubber, since materials, such as rubbers and resins, tend to swell under the presence of an aromatic compound.

Also, together with or instead of the above-mentioned reservoir 18, an air bubble trap (not shown) may be provided for moving air bubbles outside acoustic medium liquid storage space portion 15 by differences in surface tensions and specific gravities between air bubbles and acoustic medium liquid 6.

Components of ultrasound probe 1 that come into contact with acoustic medium liquid 6 are preferably formed from a silicone rubber, a fluorosilicone rubber, a fluororubber, or the like, which is resistant to swelling under an aromatic compound environment. Since materials such as rubbers or resins tend to swell under an aromatic compound environment, a sealing member (e.g., sealing member 16 for tightly bonding frame 10 and window 9), such as an O-ring or a gasket, that probably comes into contact with acoustic medium liquid 6 is preferably formed from a silicone rubber, a fluorosilicone rubber, or a fluororubber.

Also, since materials such as rubbers and resins tend to swell under the presence of an aromatic compound, adhesives (e.g., adhesive 17) that probably come into contact with acoustic medium liquid 6 are preferably epoxy, silicone, or fluorosilicone adhesives.

Further, since materials such as rubbers and resins tend to swell under the presence of an aromatic compound, resin surfaces (e.g., inner surface 19 of window 9 made of a resin) that probably come into contact with acoustic medium liquid 6 are preferably applied with coatings. For example, fluorine coatings, polyparaxylylene coatings, or inorganic film coatings are useful. Particularly, among the inorganic film coatings, when electrically conductive metal inorganic film coatings are applied, a shielding effect of external electromagnetic noise can also be obtained.

As described above, according to the embodiment, a mismatch of acoustic impedances between an acoustic medium liquid of an ultrasound probe and a living body is eliminated. Therefore, it is possible to suppress noise due to multiple reflections and obtain high-quality ultrasound diagnostic images.

What is claimed is:

1. An ultrasound probe comprising:
a piezoelectric element configured to transmit and receive an ultrasonic wave;
a casing which houses the piezoelectric element; and
an acoustic medium liquid which fills a space between the piezoelectric element and the casing, and contains an aromatic compound or a substituted derivative thereof

2. The ultrasound probe according to claim 1, wherein the acoustic medium liquid contains an aromatic compound or a substituted derivative thereof represented by the following General Formula 1: 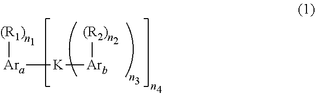
wherein Ara and Am are each independently an aromatic ring; n₁ is an integer of 0 to 4; n₂ is an integer of 0 to 3; n₃ is an integer of 1 to 3; n₄ is 0, 1, or 2; when n₄ is equal to 0, n₁ is not equal to 0; when n₄ is not equal to 0, (n₁+n₂) is not equal to 0; K is a linking group selected from the following 1) to 3)
1) a single bond,
2) a divalent group selected from the group consisting of —O—, —SO₂—, —O—(C═O)—O—, —(C═O)—, —RL-O—, —O—RL-, —O—C(═O)—RL-, —C(═O)—O—RL-, —(C═S)—, —(C═O)—O—, —NRM-, —S—, —(C═O)—NRM-, and —NRM-(C═O)—, wherein RL represents an alkylene group, an alkenylene group, an alkynylene group, or a cycloalkylene group, and RM represents a hydrogen atom or an alkyl group, and
3) a di-, tri-, or tetravalent C₁₋₁₂ saturated hydrocarbon group or a substituted group thereof; and
R₁ and R₂ are each independently a C₁₋₃₀ alkyl group or a substituted group thereof

3. The ultrasound probe according to claim 1, wherein the acoustic medium liquid has a viscosity of 22 mm²/s or lower at 40° C.

4. The ultrasound probe according to claim 1, wherein the acoustic medium liquid is benzyltoluene.

5. The ultrasound probe according to claim 1, wherein the acoustic medium liquid is 1-phenyl-1-xylylethane, 1-phenyl-1-ethylphenylethane, or a mixture thereof.

6. The ultrasound probe according to claim 1, comprising a swinging mechanism section configured to mechanically swing the piezoelectric element or a rotating mechanism section configured to mechanically rotate the piezoelectric element.

7. The ultrasound probe according to claim 6, wherein the swinging mechanism section or the rotating mechanism section includes a transmission mechanism configured to swing or rotate the piezoelectric element in tandem with a movement of the transmission mechanism, and a motor configured to drive the movement of the transmission mechanism.

8. The ultrasound probe according to claim 1, wherein a component which comes into contact with the acoustic medium liquid comprises a silicone rubber, a fluorosilicone rubber, or a fluororubber.

9. The ultrasound probe according to claim 1, comprising an acoustic medium liquid storage space portion which is tightly closed with a window which constitutes part of the casing and a frame which is a holding member, the acoustic medium liquid storage space portion being configured to retain the piezoelectric element and the acoustic medium liquid.

10. The ultrasound probe according to claim 9, comprising a sealing member which is disposed between the window and the frame, and is configured to seal the acoustic medium liquid storage space portion in a liquid-tight manner, the sealing member comprising a silicone rubber, a fluorosilicone rubber, or a fluororubber.

11. The ultrasound probe according to claim 9, wherein the window and the frame are bonded with an epoxy adhesive, a silicone adhesive, or a fluorosilicone adhesive, and the acoustic medium liquid storage space portion is sealed in a liquid-tight manner.

12. The ultrasound probe according to claim 9, comprising a reservoir configured to absorb expansion and/or contraction of the acoustic medium liquid by allowing the acoustic medium liquid to flow into and/or out of the reservoir, the reservoir being connected to the acoustic medium liquid storage space portion and comprising a fluororubber.

13. The ultrasound probe according to claim 1, wherein a surface of the casing which comes into contact with the acoustic medium liquid is applied with a coating.

14. The ultrasound probe according to claim 13, wherein the coating is a fluorine coating, a polyparaxylylene coating, or an inorganic film coating.

15. An ultrasound diagnostic apparatus comprising the ultrasound probe according to claim 1.
